(19) United States
(12) Patent Application Publication
(10) Pub. No.: US 2017/0106360 A1
(43) Pub. Date: Apr. 20, 2017

(54) ISOTHERMAL CHEMICAL PROCESS

(71) Applicant: Jay S. Meriam, Louisville, KY (US)

(72) Inventor: Jay S. Meriam, Louisville, KY (US)

(21) Appl. No.: 15/330,517

(22) Filed: Sep. 30, 2016

Related U.S. Application Data
(60) Provisional application No. 62/284,445, filed on Sep. 30, 2015.

(51) Int. Cl. B01J 35/00; C10G 9/24; B01J 23/26; C01C 3/02; B01J 25/02; C07C 5/333; C07C 253/18

(52) U.S. Cl. CPC B01J 35/0033; C07C 5/3332; C07C 5/3335; C10G 9/24; C07C 253/18; C01C 3/02; B01J 25/02; B01J 23/26; C07C 2525/02; C07C 2523/26; C10G 2300/4006

(57) ABSTRACT

Endothermic reactions (those whose heat of reaction is positive) may be controlled in a truly isothermal fashion with external heat input applied directly to the solid catalyst surface itself and not by an indirect means external to the actual catalytic material. This heat source can be supplied uniformly and isothermally to the catalyst active sites solely by conduction using electrical resistance heating of the catalytic material itself or by an electrical resistance heating element with the active catalytic material coating directly on the surface. By employing only conduction as the mode of heat transfer to the catalytic sites, the non-uniform modes of radiation and convection are avoided permitting a uniform isothermal chemical reaction to take place.

### CROSS REFERENCE TO RELATED APPLICATIONS

This application claims priority from U.S. Provisional Application Ser. No. 62/284,445 filed on Sep. 30, 2015 and is incorporated herein in its entirety.

### TECHNICAL FIELD OF THE INVENTION

This invention relates to an isothermal chemical process for conducting heat directly to a solid catalyst surface providing a uniform isothermal chemical reaction.

### BACKGROUND OF THE INVENTION

Reference to documents made in the specification is intended to result in such patents or literature cited are expressly incorporated herein by reference, including any patents or other literature references cited within such documents as if fully set forth in this specification.

In the chemical process industries, many chemical reactions take place over heterogeneous catalysts. Typically, these catalysts take the form of solid particles of widely varying sizes and shapes over which is passed the reacting fluid, a gas or gas/liquid mixed phase. All chemical reactions proceed from their initial state toward a state of thermodynamic equilibrium and the rate at which the chemical reaction takes place over the catalyst is determined by the nature of the catalyst itself (kinetics), the reaction temperature, and thermodynamic equilibrium.

The term ‘isothermal’ has a very special place in the field of chemical engineering. It represents the ideal conditions under which a preferred reaction may occur and control of the desired reaction maintained. Because all chemical reactions occur with a change in overall free energy, any conversion will result in a change in temperature. If the temperature increases or decreases, the reaction is no longer isothermal and the parameters that affect the reaction rates change. With no exchange of energy between the reacting fluid and its surroundings, the reaction is considered adiabatic.

Adiabatic performance will always be undesirable from considerations of species conversion, temperature, and equilibrium. This is because exothermic reactions will have the most favorable equilibrium conversion at low temperatures and endothermic reactions will have the most favorable equilibrium at high temperatures. The adiabatic temperature of either reaction proceeds in the opposite direction from the temperature of most favorable equilibrium. These basic thermodynamic principles are well understood. What follows is understood to apply to chemical reactors in which a heterogeneous catalyst is employed to accelerate the reaction rates to commercially acceptable levels.

In a reactor employing heterogeneous catalysts, kinetics also play an extremely important role and the temperature relationship to kinetics is vital. As heterogeneous catalysts are employed to accelerate the rate of reaction to its equilibrium state, the overall rate of a chemical reaction a catalytic surface is primarily dependent on three factors. The first factor is the thermodynamic factor which will accelerate the reaction if the state of the reactants is “far” from its equilibrium state. As the reaction approaches its equilibrium values, the forward rate of the reaction over the catalyst will be reduced. Of course, when the reaction reaches equilibrium the forward and reverse rates of reactants and products become the same and no overall rate is observed.

The second factor to influence the rate of reaction is the adsorption energy of reactants and products on the catalyst surface. The more strongly the reactants are adsorbed onto the catalyst surface, the slower the reaction will become.

The third factor to influence the reaction rate is the temperature of the reaction which follows the form:

Ln(rate)=Ln(A)−Ln(Eₐ/RT)

This is well known as the Arrhenius equation where A and Eₐ are constants depending on the nature of the specific catalyst. A is a constant determined by experimentation and R is the standard gas constant (1.987 cal/gmol−° C.). The constant, Eₐ, is called the activation energy of the reaction and is again a property of the specific catalyst for a particular reaction as determined by experimentation.

If the temperature is invariant (truly isothermal), conditions of states and the reaction rates of chemical reactions can be optimized in commercial units to produce the maximum amount of product (and least byproducts) for the minimum capital investment. However, if the temperature of the reaction at the surface of the catalyst varies because of thermodynamic cooling or non-uniform rates of applied external heat, optimum reaction rates cannot be achieved. This is the need for a truly isothermal reactor in which all of the chemical reactions take place on the catalytic sites at precisely the same temperature.

A chemical reactor operating in a truly isothermal manner would provide for the best possible conversion (equilibrium conversion) at a given temperature. For exothermic reactions, this would require that at every point of reaction where heat is evolved there is a physical mechanism to remove exactly the same amount of heat from the fluid. Likewise, for an endothermic reaction, the exact heat of reaction would need to be supplied from an external source.

There have been many attempts to achieve this ideal each in its own way supplying or removing heat from the reacting fluid by some mechanical means. Some of the most common methods include tubular reactors in which tubes are inserted axially or radially within the catalyst bed each containing a separate fluid flow of a different temperature to affect heat transfer through the tube to the catalyst section whereby heat is transferred into or out of the catalyst bed primarily by convection. Conversely, the reacting fluid and catalyst may be inside the tubes and with heat transfer taking place to or from a fluid external to these reactor tubes.

Two such applications would be a TVA ammonia synthesis converter wherein cooling tubes within the bed of ammonia synthesis catalyst remove a portion of the generated heat from the exothermic ammonia synthesis reaction. With a cooler medium flowing through the tubes, heat is transferred from the reacting fluid in the catalyst bed by convection and conduction to the coolant.

A second common application for endothermic reactions is the tubular reactor for steam-hydrocarbon reforming. In this application, the highly endothermic reaction takes place over catalyst within the tubes and superheated gas external to the reactor tubes supplies heat to the reacting medium, again by convection, conduction, and radiation. In both of these examples, there exists a high degree of heat transfer in the redial direction creating hot catalyst and reacting fluid near the surface the exchange surface and a much different catalyst and reacting fluid at any distance from the heat transfer surface. In the case of steam reformers with tube inside diameters of 10 cm, the radial temperature differential across the catalyst within the tubes is estimated to be nearly 20-25 degrees Celsius. In terms of reaction kinetics, this differential temperature can strongly affect reaction rates. Although tubular reactors as shown in FIG. 1, are often referred to as ‘isothermal’ reactors, in neither of these reactor designs does the operation significantly approach isothermal conditions. Truly isothermal conditions occur only when the surface temperature of the heterogeneous catalyst is of uniform temperature at all points where reaction occurs.

Maintaining the temperature of each catalyst particle exposed to the reacting fluid is a difficult task. For an endothermic reaction, heat must be supplied from an external source in such a way as to maintain the surface temperature of the catalyst at exactly the same temperature even as reaction is occurring and heat of reaction is being absorbed by the reacting fluid. Supplying the heat by convection is not an option because convective heat transfer must rely on the absorption of heat from an external source which cannot be done without a radial temperature profile being created.

It cannot be accomplished by radiative heat transfer because radiative heat is directional in nature. For example, a hot plate external to the catalyst would radiate heat from every point on the surface of the plate to the exposed catalytic points on the catalysts' surface to which there is a direct line of sight. FIG. 2 illustrates the complexities of heat transferred from different surfaces to each other. Each area at its own temperature (Aₖ, Tₖ) will radiate heat (q″ₖ) toward every other point on every other surface to which it has a direct line of sight.

Most of the catalyst activity sites are well within the porous structure of the catalyst which will not have a line-of-sight to the radiative source of heating. Hence, radiation from an external cannot uniformly heat the catalyst surface and maintain the temperature as heat is removed by the endothermic reaction.

The only method of uniform heating of the catalytic active sites is by thermal conductive heating from a source which is the active catalyst itself or from a catalyst carrier which directly supports the active catalyst particles. Uniformity of heating the individual catalyst particles is essential for isothermal reaction conditions.

Attempts to actually achieve these conditions have been rare and mostly misdirected. In recent times, there has been a novel attempt to supply heat directly to a heterogeneous catalyst for an endothermic process that is potentially more effective than the tubular reactors. U.S. Pat. No. 5,958,273 by Koch et al. for an Induction Heated Reactor Apparatus which issued in Sep. 28, 1999 and is incorporated herein shows in FIG. 3 an apparatus having a reaction zone having an inlet port and an outlet port. The reaction zone contains an array of electrically conductive catalyst entities, and is in close proximity to an induction heating device. An external source of alternating current electrical power is connected to an induction heating device so as to create a region of high intensity alternating magnetic field throughout the reaction zone, thereby heating said catalysts entities substantially uniform. This work utilizes a generated magnetic field from electric coils outside the reactor walls which imparts magnetic energy to an electrically conductive catalyst within the reactor. Although truly innovative, the magnetic field is itself non-uniform by its very nature and a radial temperature profile will be seen in the catalyst bed.

The application of this work was to supply the necessary energy for the endothermic reaction to produce hydrogen cyanide from ammonia and methane. This reaction is a common method to manufacture cyano compounds such as HCN and acrylonitrile, all of which are endothermic. Although no data are shown for this operation, the non-uniform radial temperature profile from the magnetic field will kinetically enhance side reactions in this complex mechanistic reaction. The very advantage of an isothermal reactor is to maximize the conversion of desired products and minimize by-product make.

This method of heating the catalyst bed can only be applied to catalysts comprised of magnetic conductors such as reduced metals in their zero valance state. There are many applications for non-magnetic catalysts which could not employ this method at all.

In a totally unrelated attempt at improving the function of automotive exhaust catalyst, the work of Brunson and U.S. Pat. No. 8,911,674 by Yoshioka, et. al., reveals a method to heat the exhaust catalyst more quickly to its operating temperature. This effort is necessary because the catalytic converter in automobiles needs to be at elevated temperatures for proper operation and does not reach that temperature until the hot exhaust gases themselves heat the catalyst by convective heat transfer. Because this requires a number of minutes to achieve, pollutants are introduced to the environment over that time. Yoshioka, et. al., formed the automotive catalyst carrier from an electrically conductive material such as metal or silicon carbide. These materials serve as a support of the active catalyst materials, in this case platinum particles.

The work by Brunson shows simply the electrical heating of a metal surface closely associated with a catalyst external to the heating element. Effectively, this embodies heating the catalyst by external means and the axial and radial temperature gradients that follow from this form of heating. The similar work of Yoshioka furthers the application to include electrical resistance heating of the catalyst carrier material itself.

There are a number of undisclosed elements in the Yoshioka work. First is the nature of the electrical current used to heat the catalyst carrier by resistance heating. If alternating current is used (from the automotive alternator) the temperature of the catalyst will not be uniform much the same as the catalyst bed in the Koch work. However, if direct current is used (from the auto battery), the catalyst carrier (and catalyst) would be more uniformly heated. Yoshioka does not take advantage of securing the electrical connection to the total exposed catalyst support, again creating non-uniformity of temperature within the overall catalyst structure even using direct current. Complete uniformity of radial and axial temperatures are required for true isothermal behavior and the benefits to be derived therefrom.

It is also important to understand that both Brunson and Yoshioka apply their technology simply to quickly heat the catalyst converter to operating temperature and that the reaction accelerated by this catalyst is an exothermic one. There is no teaching or appreciation of this method or in the prior art that a catalyst or catalyst carrier heated by uniform electrical resistance heating can be used to conductively supply exactly the heat of an endothermic reaction and, in this way, function in a truly isothermal manner.

### SUMMARY

Endothermic reactions (those whose heat of reaction is positive) may be controlled in a truly isothermal fashion with external heat input applied directly to the solid catalyst surface itself and not by an indirect means external to the actual catalytic material. This heat source can be supplied uniformly and isothermally to the catalyst active sites solely by conduction using electrical resistance heating of the catalytic material itself or by an electrical resistance heating element with the active catalytic material coating directly on the surface. By employing only conduction as the mode of heat transfer to the catalytic sites, the non-uniform modes of radiation and convection are avoided permitting a uniform isothermal chemical reaction to take place.

A method for carrying out isothermal endothermic chemical reactions over heterogeneous catalysts employing electrical resistance heating of an electrically conductive catalytic material as the heat source for the endothermic process. The electrical resistance heating device is in direct physical contact with the active heterogeneous catalytic agents the outer surface of which contacts the reacting fluid. A wash coat or combination of wash coats lies between an electrical resistance heating device and the active heterogeneous catalytic agents. The electrical resistance heating device is heated by means of direct electrical current.

The present invention provides a process for the manufacture of unsaturated hydrocarbons directly from saturated hydrocarbons in which the electrical resistance element is silicon carbide, graphitic carbon, or a metal from the transition series of elements in the Periodic Table of the Elements.

The present invention provides a process for manufacture of unsaturated hydrocarbons directly from saturated hydrocarbons in which the catalyst is a mixture of chromium oxides wherein the valence of the chromium is +2 to +6.

The present invention provides for a process for manufacture of chemical products which undergo endothermic reactions for their manufacture. This includes specifically direct dehydrogenation of hydrocarbons (paraffinic or olefinic) to less saturated products and the reaction of ammonia with hydrocarbons to produce cyanic compounds (R—CN where R is hydrogen or an organic group).

Other objects, features, and advantages of the invention will be apparent with the following detailed description taken in conjunction with the accompanying drawings showing a preferred embodiment of the invention.

### BRIEF DESCRIPTION OF THE DRAWINGS

A better understanding of the present invention will be had upon reference to the following description in conjunction with the accompanying drawings in which like numerals refer to like parts throughout the several views and wherein:

FIG. 1 is a view of a prior art conventional tubular reactor;

FIG. 2 shows the complexities of heat transferred from different surfaces to each other by prior art processes;

FIG. 3 shows the complexities of heat transferred from different surfaces to each other by prior art processes;

FIG. 4 is shows a prior art induction heated reactor having a reaction zone containing an array of electrically conductive catalyst entities in close proximity to an induction heating device requiring an external source of alternating current electrical power connected to an induction heating device so as to create a region of high intensity alternating magnetic field throughout the reaction zone, thereby heating the catalysts;

FIG. 5 is a graph showing the typical relationship between the overall Yield and the temperature of operation;

FIG. 6 shows an electrically conductive Raney Nickel rod of catalytic material;

FIG. 7 shows an electrically conductive silicon carbide rod having active catalyst particles exposed directly on the surface of the rod; and

FIG. 8 shows a silicon carbide rod 14 including an Al₂O₃ washcoat and active catalyst particles exposed on the surface of the catalyst rod.

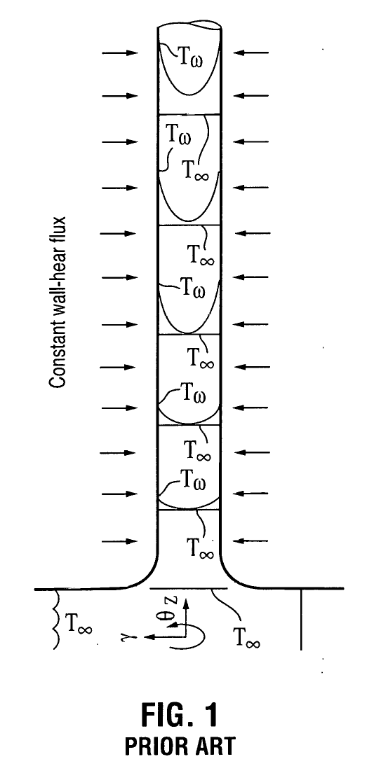

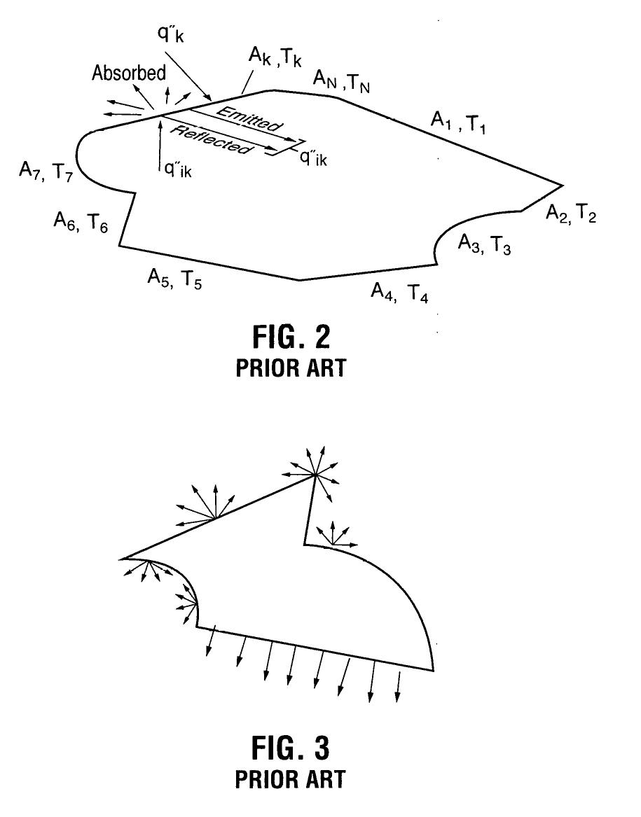

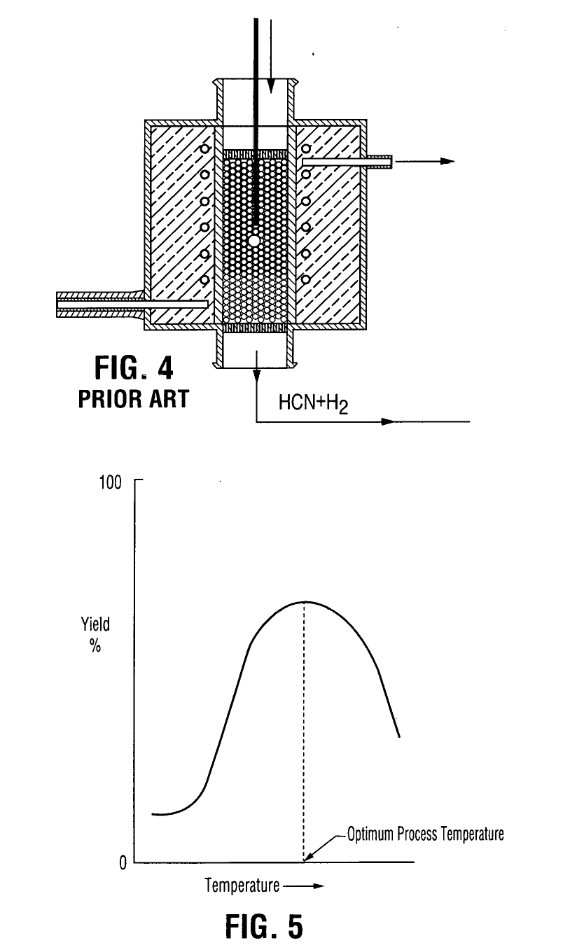

### DETAILED DESCRIPTION OF THE PREFERRED EMBODIMENT

For the purposes of promoting an understanding of the principles of the invention, reference will now be made to the embodiment illustrated in the drawings and specific language will be used to described the same. It will nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

As stated earlier, the advantage of truly isothermal reactors lies in the thermal control of the catalyst and reacting fluid to maximize effectiveness of reaction conversion and the minimization of by-products.

When more than one reaction can take place over the catalyst conversion can be defined as the amount of reactant consumed in the reaction divided by the original amount of reactant:

Conversion=(mols inlet−mols outlet)/tools inlet

If all of the mols of reactant are converted to the desired product, it is said that the reaction is 100% selective. However, if additional or side reactions can occur, the Selectivity will be less than 100%. The Selectivity is defined as the mols of reactant going to product divided by the total mols converted.

Selectivity=((mols product out)/(mols reactant converted))×100

The arithmetic product of Conversion and Selectivity is termed Yield.

Yield−Conversion X Selectivity/100

In a commercial operation, the total product produced is defined as the Yield and the highest Yield can be achieved only under isothermal conditions because temperature gradients will lower Conversion to maintain a specified Selectivity or they will decrease Selectivity if Conversion is maintained by higher temperature. This is illustrated in FIG. 5 showing the typical relationship between the overall Yield and the temperature of operation.

In FIG. 5, the rising Yield is due to a more rapid increases in Conversion for the principle product. Yield will reach a maximum at temperature tₘₐₓ and will reduce with additional temperature because the by-product make increases faster than product increase. For any particular reaction of this sort, it is obvious that any temperature other than tₘₐₓ will result in lower Yield. This applies equally well to any process in which there is a temperature gradient, ΔT, common to all other reactor designs.

In some commercial applications, by-product make is of minimal importance. Such an example is the steam reforming of hydrocarbons wherein light hydrocarbon molecules are reacted with steam at high temperatures to produce a mixture of hydrogen, carbon monoxide, carbon dioxide, and steam. This is a highly endothermic reaction occurring over very active nickel based catalyst and is usually performed in a tubular reactor. Because the reaction is endothermic, the equilibrium conversion to product is greater at higher temperatures.

A possible side reaction is the Boudouard Reaction, two molecules of carbon monoxide reacting to form one molecule of carbon dioxide and one molecule of carbon. Because the elemental carbon formed is a solid, it will build up on the catalyst surface and poison the ability of the catalyst to function. Fortunately, this reaction does not commonly occur in commercial reactors due to the temperature limitation of the reactor tubes (a thermodynamic limitation) and the high partial pressure of steam (a kinetic limitation) employed to further the principle reaction to more favorable equilibrium.

Because steam reforming can be performed with only small regard to carbon formation, this does not apply to all applications. Specifically, the direct dehydrogenation of hydrocarbons suffers strongly from carbon by-product at commercial operating conditions.

There are several commercial processes for the direct dehydrogenation of hydrocarbons to olefins all of which must supply the necessary heat of reaction from an external source. For example, the Oleflex® and Catofin® processes supply heat by means of externally heating the catalyst and subsequent contact with the reacting hydrocarbon fluid. Because the catalyst must be heated to high temperatures (>650° C.), hydrocarbon contact with the catalyst produces coke as a by-product. Heat transfer occurs between the hot catalyst particles and the fluid until all of the externally added heal is consumed by the heat of reaction.

Under commercial reactor conditions, the dehydrogenation reaction will reach equilibrium at 550-560° C. at the reactor exit but the solid coke is formed in the layers of the catalyst bed which are above 600° C. This is vitally important because it shows that for an isothermal operation at temperatures below 600° C., the process could be carried out on a continuous basis without the buildup of deleterious coke. If, for example, a truly isothermal reactor of the invention were employed for this reaction, not only could the reactor operated in continuous mode but the outlet temperature could likely be increased to 580-590° C. and a significant gain in conversion could be achieved because of the more favorable equilibrium.

The STAR® process operates in a tubular reactor at lower temperatures limiting the conversion to olefin but reducing the amount of coke that is formed. Coke is not eliminated, only reduced along with the conversion. The residual coke that is formed must be removed from the catalyst in a separate process (usually burned off in-situ). As earlier stated, tubular reactors operated for endothermic reactions can have severe temperature differentials in the catalyst bed and a radial temperature profile that can vary 20-25° C. Hence, in all of the commercial processes for direct dehydrogenation of hydrocarbons, solid coke is formed and lowering the operating temperature to reduce the amount of coke formation leads to less than acceptable product.

All three of the commercial processes suffer from the coke formation caused by external heating features that must be hotter than the final temperature of the fluid. Contact of the hot catalyst above the 650° C. will in every case cause irreversible coke to be formed.

The ideal reactor for this type of application is a truly isothermal reactor which can control the temperature at a uniform level to maximize the conversion of hydrocarbon to olefin yet at a temperature sufficiently low that coke does not form. Such a temperature can be less than 600° C. where conversion is at least that of current technology and potentially higher. Isothermal temperatures below 600° C. become very attractive economically as the Yield will not suffer from Selectivity loss at higher temperatures.

Under such conditions, the process can be continuous without the need for cyclic regeneration as in the CATOFIN® example or a separate reactor for burning off coke and heating the catalyst as in the OLEFLEX® process. Because the temperatures can be easily controlled below the coke forming temperature, it can avoid any coke deposition as in the Star process because there are no hot tube walls to heat the catalyst bed.

In the above examples, a desired continuous process would suffer catastrophic failure if the side reaction (coking) were to occur on the surface of the catalyst. Under these circumstances, for maximum conversion of saturated hydrocarbon to olefin, the precise kinetics of the principle and side reactions must be known. Referring to the explanation of kinetic control (vida infra), it is important to characterize the activation energies, Eₐ, for each reaction. This is the important characteristic of a catalyst which responds to the rate effect by temperature. With the exact knowledge of the individual reaction kinetics, dehydrogenation of hydrocarbons can be effectively carried out in the temperature region where the reaction rate of coke formation is negligible and the conversion to olefin is maximized.

### Examples of Application of the Isothermal Chemical Process

The following examples describe preferred embodiments of the invention. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims which follow the examples. In the examples all percentages are given on a weight basis unless otherwise indicated.

For a truly “isothermal” reaction to take place, the surface of the catalyst particles where adsorption, reaction, and desorption of reactants and products occurs must be continuously at the same temperature. These small catalytic particles where chemical reaction occurs are well known in the industry as “active sites” and I have found the only way to achieve such a constant temperature at all of the catalyst surfaces and active sites is by means of an electrical resistance element which serves as the catalyst itself or which serves as a physical support for the individual catalyst active sites. In this way, an applied electrical potential across the electrical resistance element will increase the temperature of the element and any catalytic active sites directly attached to the element to a constant and controlled temperature. It is then a simple application of the appropriate electrical current to maintain the temperature of the catalyst surface, where all of the reaction is taking place, at a fixed, desirable temperature. This method relies on the uniformity of conductive heat transfer directly to the catalyst active sites and does not rely on the irregular and inconsistent heat transfer mechanisms of radiation and convection.

### Example 1

Example 1 illustrates an application where the electrical resistance heating element acted as the catalyst itself and no other components were required except the applied electrical current.

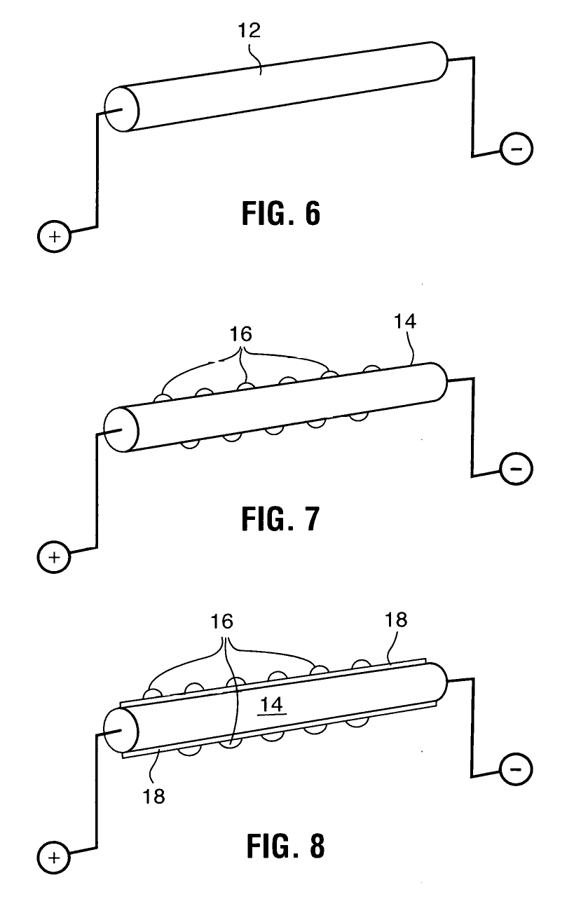

The electrically conductive rod 12 of catalyst material shown in FIG. 6 is used as an electrical resistance element. For example, the conductive rod 12 may comprise solid nickel metal or a RANEY NICKEL sometimes referred to a “skeletal catalyst” or “sponge-metal catalyst” which is a product produced by W.R. Grace and Company. RANEY NICKEL is a proven catalyst made by forming an alloy of aluminum and nickel metals. The aluminum is leached from the alloy with a caustic solution leaving only metallic nickel with a high B.E.T. surface area. Because nickel is a conductor of electricity, an applied voltage across a nickel surface will serve to increase the temperature of the catalytic nickel uniformly.

More particularly, the Ni—Al alloy is prepared by dissolving nickel in molten aluminum followed by cooling (“quenching”). Depending on the Ni:Al ratio, quenching produces a number of different phases. During the quenching procedure, small amounts of a third metal or promoter, such as zinc or chromium, are added to enhance the activity of the resulting catalyst. The promoter changes the mixture from a binary alloy to a ternary alloy, which can lead to different quenching and leaching properties during activation. In the activation process, the alloy, usually as a fine powder, is treated with a concentrated solution of sodium hydroxide forming sodium aluminate (Na[Al(OH)4]). The surface area of Raney nickel tends to decrease with increasing leaching temperature due to structural rearrangements within the alloy that may be considered analogous to sintering, where alloy ligaments would start adhering to each other at higher temperatures, leading to the loss of the porous structure. During the activation process, Al is leached out of the NiAl3 and Ni2Al3 phases that are present in the alloy, while most of the Al remains, in the form of NiAl. The removal of Al from some phases but not others is known as “selective leaching”. The NiAl phase provides the structural and thermal stability of the catalyst. The catalyst is resistant to decomposition. Raney nickel is available as a finely divided gray powder. Each Microscopic particle of powder is a three-dimensional mesh, with pores of irregular size and shape of which the vast majority are created during the leaching process. Raney nickel is notable for being thermally and structurally stable, as well has having a large BET (Brunauer-Emmett-Teller) surface area. These properties are a direct result of the activation process and contribute to a relatively high catalytic activity. The surface area is typically determined via a BET measurement using a gas that will be preferentially adsorbed on metallic surfaces, such as hydrogen. Using this type of measurement, almost all the exposed area in a particle of the catalyst has been shown to have Ni on its surface implying a large surface is available for reactions to occur simultaneously, which is reflected in an increased catalyst activity.

A high catalytic activity, coupled with the fact that hydrogen is absorbed within the pores of the catalyst during activation, makes Raney nickel a useful catalyst for many hydrogenation reactions. Its structural and thermal stability (i.e., it does not decompose at high temperatures) allows its use under a wide range of reaction conditions.

Raney Nickel is a useful catalyst for many reactions both exothermic (hydrogenation of hydrocarbons) and endothermic (steam reforming of hydrocarbons) reactions. Of course, it serves no purpose to heat the catalyst for exothermic reactions but for the example reaction of steam reforming of hydrocarbons, it has special application. This special application applies to the temperature limitations of traditional “isothermal” tubular reformers. In these units, the process gas (steam and hydrocarbon) pass through the interior of long steel tubes containing catalyst particles. These catalyst particles typically have a refractory base onto which have been deposited very small particles of nickel metal.

An advantage to the use of electrical resistance heating is that much higher temperatures are possible than can be applied through steel tubes. Outer wall temperatures of the containment tubes are limited for the highest grade stainless steel to about 1000° C. Of course, for the endothermic steam reforming reaction, much more favorable equilibrium conversions lie at higher temperatures and much higher temperatures are easily achievable with electrical resistance elements. For this application, electrically heated nickel or Raney Nickel rods could be operated at temperatures up to the softening point of the metal although an increase in outlet temperature of 50-100° C. would be much for favorable for conversion.

### Example 2

Example 1 illustrated an application where the electrical resistance heating element acted as the catalyst itself and no other components were required except the applied electrical current.

In commercial catalytic reactors, this is rarely the case. Most active catalysts are themselves not electrical conductors in the same category as metallic nickel or perhaps another catalytically active metal (Pt, Pd, etc). Many catalysts are combinations of metal oxides, chlorides, or sulfides and are effectively electrical insulators.

FIG. 7 shows an electrically conductive silicon carbide rod 14 having active catalyst particles 16 exposed directly on the surface of the rod.

For such materials, it is necessary to precipitate these active components onto the surface of an electrical conductive material, which could be a metal as previously described or, more practically, an element such as silicon carbide. Applying active catalyst to the surface of a solid silicon carbide rod (or other shape) permits electrical current to be passed through it creating heat by electrical resistance. Because the outer temperature of the electrical resistance heating element is constant, the temperature of the catalytic active sites on the surface is constant as well. The isothermal temperature of the desired reaction is then easily controlled by simply varying the electrical current through the electrical resistance heating element.

In addition to silicon carbide, most metals in their metallic state and electrically conductive forms of carbon may also be used as electrical heating elements upon which catalytic elements may be adhered.

The heat of chemical reaction on the catalyst surface can be supplied and balanced by the electrical heating resistance element such that the surfaces of the catalytic components remain at constant temperature.

This is the very definition of an isothermal reaction and has application to many chemical reactions including the example given above for direct dehydrogenation of hydrocarbons. Because an effective catalyst for this process is Cr₂O₃ (usually deposited on a refractory support), the use of SiC rods as a support for the Cr₂O₃ is ideal. Cr₂O₃ itself is not electrically conductive and could not be used in the application of Brunson but can easily be deposited onto the surface of a SiC rod heated by electrical resistance. Because the SiC will be at a constant temperature, the catalytic material directly adhering to the surface will also be at constant temperature and truly isothermal reaction will occur with the benefits described heretofore.

### Example 3

FIG. 6 shows a silicon carbide rod 14 including an Al₂O₃ washcoat 18 and active catalyst particles 16 exposed on the surface of the catalyst rod.

Catalytic components, such as finely divided metal crystallites of elemental nickel for the steam reforming reaction, are more active when dispersed on an inert carrier material such as aluminum oxides or silicon oxides. Because the nickel crystallites are more active on alumina, it is clear that a layer of aluminum oxide may also be chemically precipitated on the surface of an electrical resistance heating element in a uniform layer and the nickel added onto the aluminum oxide in like fashion. This, in effect, creates what is known in the industry as a “wash coat” of support material on another material more desirable for mechanical integrity or enhanced catalytic activity. Using the electrical resistance heating element as the base material, wash coats of other catalyst support materials can be applied as is common practice in the art. This in no way alters the efficacy of the invention. If the wash coat contributes thermal resistance to the flow of conductive heat from the electrical resistance heating element to the catalyst particles, the temperature of the active catalyst particles may be maintained simply with addition electrical current through the electrical resistance heating element.

The foregoing detailed description is given primarily for clearness of understanding and no unnecessary limitations are to be understood therefrom, for modification will become obvious to those skilled in the art upon reading this disclosure and may be made upon departing from the spirit of the invention and scope of the appended claims. Accordingly, this invention is not intended to be limited by the specific exemplifications presented herein above. Rather, what is intended to be covered is within the spirit and scope of the appended claims.

I claim:

1. A method comprising the steps of:
conducting an isothermal endothermic chemical reactions over a heterogeneous catalysts;
and heating an electrically conductive catalytic material by electrical resistance whereby said electrically resistance comprises the only heat source for the endothermic process.

2. The method of claim 1, further including the step of:
employing an electrical resistance heating device in direct physical contact with an active heterogeneous catalytic agent disposed on the outer surface of said electrically conductive catalytic material; and
said electrically conductive catalytic material directly contacting a reacting fluid.

3. The method of claim 1, further including the step of:
applying at least one wash coat between said electrical resistance heating device and the said active heterogeneous catalytic agent.

4. The method for carrying out isothermal endothermic reactions as in claims 1, wherein the electrical resistance heating device is heated by means of direct electrical current.

5. The method for carrying out isothermal endothermic reactions as in claims 2, wherein the electrical resistance heating device is heated by means of direct electrical current.

6. The method for carrying out isothermal endothermic reactions as in claims 3, wherein the electrical resistance heating device is heated by means of direct electrical current.

7. The process for manufacture of unsaturated hydrocarbons directly from saturated hydrocarbons according to claims 1 in which the electrical resistance element is Silicon Carbide, graphitic carbon, or a metal from the transition series of elements in the Periodic Table of the Elements.

8. The process for manufacture of unsaturated hydrocarbons directly from saturated hydrocarbons according to claims 1 in which the catalyst is a mixture of chromium oxides wherein the valence of the chromium is +2 to +6.

9. The process for manufacture of chemical products which undergo endothermic reactions for their manufacture. This includes specifically direct dehydrogenation of hydrocarbons (paraffinic or olefinic) to less saturated products and the reaction of ammonia with hydrocarbons to produce cyanic compounds (R—CN where R is hydrogen or an organic group).
